(19) United States
(12) Patent Application Publication
(10) Pub. No.: US 2006/0110476 A1
(43) Pub. Date: May 25, 2006

(54) Dietary foodstuff for positively influencing cardiovascular health

(76) Inventors: Bernd Haber, Mainz (DE); Thomas Kiy, Frankfurt (DE); Stephan Hausmanns, Wiesbaden (DE); Matthias Ruesing, Koeln (DE)

Correspondence Address: PROPAT, L.L.C., 425-C SOUTH SHARON AMITY ROAD, CHARLOTTE, NC, 28211-2841, US

(21) Appl. No.: 10/538,903

(22) PCT Filed: Dec. 22, 2003
(86) PCT No.: PCT/EP03/14714

§ 371 Date: Jun. 14, 2005

(30) Foreign Application Priority Data
Dec. 24, 2002 (DE) 10261061.4

(51) Int. Cl. A61K 36/48; A61K 31/685; A61K 31/225

(52) U.S. Cl. 424/757; 514/78; 514/547; 514/560

(57) ABSTRACT

This invention relates to a dietary foodstuff containing at least one carob product, particularly water-insoluble carob fibers, and at least one n-3 fatty acid. The invention also relates to a method for producing active substance combinations of this type and to the use thereof.

The invention relates to a dietetic food comprising at least one cholesterol-reducing carob product, in particular carob fibers, and at least one n-3 fatty acid, for reducing the cholesterol level and the triglyceride level and also to a positive shift of the HDL/LDL cholesterol ratio. The invention further relates to a method for producing such dietetic foods and also to their use.

In the context of an unbalanced diet, broad sections of the population display an elevated content of blood fat values, in particular blood cholesterol values. A cholesterol value of greater than 200 mg/dl, in particular LDL cholesterol values greater than 130 mg/dl, is considered one of the principal risk factors for cardiovascular disorders. Therefore, therapeutic treatment in the case of significantly increased cholesterol values, in particular LDL cholesterol, and increased blood fat values, is urgently necessary. To date, various approaches to a solution have been described for this. In addition to switching lifestyle and nutritional habits, which is of generally only slight efficacy, a number of special active compounds have been developed which intervene in different ways in the intake and metabolism of cholesterol. These are, inter alia, pharmacologically active substances such as statins (see, eg, U.S. Pat. No. 4,231,938; U.S. Pat. No. 4,447,84; U.S. Pat. No. 4,346,227), inhibitors of bile acid resorption (see, eg, U.S. Pat. No. 5,998,400; U.S. Pat. No. 6,277,831; U.S. Pat. No. 6,221,897) or bile acid sequestrants (see, eg, U.S. Pat. No. 4,027,009). All these active compounds must be taken under medical supervision and monitoring.

The active compounds can also comprise cholesterol-reducing agents isolated from plant sources. Here, primarily, the cholesterol-reducing agent of a group of plant sterols, in particular phytosterols, phytostanols and the esters of said compound classes (see, eg, WO-A-96/38047, WO-A-99/56558, U.S. Pat. No. 6,087,353) may be mentioned. Primarily the latter, however, are unsuitable for consumption by all population groups (eg exceptions for pregnant women or infants) and are frequently restricted in their application.

In contrast, there are food components which have demonstrated repeatedly that, when consumed sufficiently, they can significantly reduce the risk of cardiovascular disorders, in particular also by reducing increased cholesterol levels. It is generally known that a high-fiber diet, compared with a low-fiber diet, is associated with a lower risk of cardiovascular disorders. In addition to whole-grain cereal (wheat, oats, barley, rye, but also cereal brans such as oat bran, rice bran, wheat bran, soy bran etc.) which is generally high-fiber, other fibers can also make a contribution to reducing cardiovascular risk and the increased cholesterol level. For instance, a number of water-soluble fibers, e.g. β-glucan (from oats or barley), psyllium, pectin or guar gum, demonstrate a reducing action on the blood cholesterol level (Brown et al. 1999; Am. J. Clin. Nutr. 69: 30-42).

In addition, water-insoluble carob fibers are known as food components, for example those produced by a method according to EP-A-0 616 780, which can significantly reduce serum cholesterol values, in particular LDL cholesterol (Zunft et al. 2001, Adv. In Ther. 18: 230-36). The HDL value remains constant here, so that the important LDL/HDL ratio is shifted toward the “good cholesterol”, and thus the arteriosclerosis risk decreases. The marked action of this insoluble, non-viscous preparation was the more surprising, since such reductions in cholesterol generally only occur in the case of viscous, soluble fibers.

Further food components which can contribute to a significant reduction in the risk of cardiovascular disorders comprise n-3 fatty acids. It is known that in most industrial countries the supply with n-3 fatty acids is deficient. In contrast, in particular the total fat content in the diet and the supply of saturated fatty acids and n-6 fatty acids is too high. This is based on a change in our food composition which took place primarily in the last approximately 150 years and which is correlated with the occurrence of various chronic disorders (of civilization), in particular cardiovascular disorders, the principal cause of death in industrial countries. A multiplicity of studies has found in the interim that the targeted increase in the supply of n-3 fatty acids, in particular all-cis-5,8,11,14,17-eicosapentaenoic acid (EPA) and all-cis-4,7,10,13,16,19-docosahexaenoic acid (DHA) can significantly reduce the cardiovascular risk [GISSI-Prevenzione Investigators (Gruppo Italiano per lo Studio della Soprawivenza nell'Infarto miocardico), Dietary supplementation with n-3 polyunsaturated fatty acids and vitamin E after myocardial infarction: results of the GISSI-prevenzione trial. Lancet. 1999; 354: 447-455; Burr et al., Effects of changes in fat, fish and fibre intake on death and myocardial reinfarction: diet and reinfarction trial (DART), The Lancet, 1989, 757-761]. Correspondingly, many different organizations (WHO, FAO, AHA, ISSFAL, British Nutrition Foundation and many others) recommend significantly increasing the supply of n-3 fatty acids. Here (depending on recommendation), a deficiency of supply of at least 0.5 to 1.5 g of n-3 fatty acids is found. The most recommendations relate here to the supply of n-3 fatty acids (in particular DHA and EPA) by regular consumption (at least 2×weekly) of fatty sea fish. Although the beneficial effects on reduction of cardiovascular risk by n-3 fatty acids are often not clear in detail, they are primarily associated with beneficial effects on some of the main risk factors for cardiovascular disorders such as arteriosclerosis, high blood pressure, plasma triglyceride level, arrhythmias and heart frequency variability. Interestingly, the n-3 fatty acids appear to have no effect, or only a slight effect, on a further main risk factor, the cholesterol level. At all events, a slight shift in the LDL/HDL ratio toward the “good cholesterol” is being discussed (Gylling and Miettinen, Curr Control Trials Cardiovasc Med 2001, 123-128).

However, the effects which can be achieved with all these food components are significantly below those which are achieved with therapeutic active compounds, and are thus far lower than desirable. Even if a diet enriched with fibers, in particular carob fibers, can make a contribution toward controlling the cholesterol level and the blood fat values, in many cases, in particular in the case of very high cholesterol levels (total cholesterol >300 mg/dl) it is insufficient for a lasting reduction. Likewise, a diet enriched with n-3 fatty acids, in particular with all-cis-9,12,15-octadecatrienoic acid (ALA), EPA and DHA, can make a valuable contribution to general reduction of cardiovascular risk and to improving general health, in many cases, in particular in the case of an increased cardiovascular risk (eg after a heart attack), this alone is not sufficient.

A cholesterol-reducing and blood-fat-reducing interaction between carob products and n-3 fatty acids is not known. However, there are indications that viscous fibers such as pectin can have, with n-3 fatty acids, a synergistic effect in cholesterol reduction (V. Bartz 2002, Ernährung & Medizin 17, 149-150). Since carob products, in particular carob fibers, are not viscous, a cholesterol-reducing and blood-fat-reducing interaction is not obvious, certainly not a synergistic interaction. For example, an antagonistic action has even been described of the water-insoluble fibers of the carob fruit flesh with the viscous dietary fiber carob seed meal (Peres-Olleros et al. 1999; J. Sci. Food Agric. 79, 173-178).

The purely pharmacological cholesterol-reducing agents have the disadvantage that, to achieve the therapeutic purposes, considerable concentrations must sometimes be used. In this case, unwanted partially life-threatening side effects can occur. Furthermore, saturation effects are known which mean that, with an increased intake of the active compound, only slight additional reductions of the cholesterol level are achieved. A further disadvantage are the high costs which occur in long-term therapies using the usually very expensive pharmacological cholesterol-reducing agents. In the case of cholesterol-reducing agents isolated from plant sources (e.g. phytosterols), there are quantitative limits to avoid unwanted side effects.

There is therefore still a requirement for cholesterol-reducing and blood-fat-reducing dietetic foods which can make a beneficial contribution to the daily diet in this respect.

This object is achieved by providing a dietetic food for reducing the cholesterol level and the blood fat level which comprises at least one carob product, in particular carob fibers, and at least one n-3 fatty acid. When the inventive dietetic food is administered, in addition to the above described effect of the total cholesterol reduction and reduction of the blood fat values, a shift of the ratio of HDL and LDL to the “good” HDL cholesterol occurs.

In addition, this synergistic reduction in the cholesterol level and the reduction in the blood fat values by the inventive dietetic foods are advantageously supplemented by the known beneficial effect of n-3 fatty acids on the cardiovascular system (see above).

Independently of the above described beneficial effects on cardiovascular health, the inventive dietetic foods achieve an additional beneficial effect on health by means of an increased supply of n-3 fatty acids. DHA, which is preferably used according to the invention, plays a particular role here. In addition, the inventive active compound combination can compensate for a depletion of the body in essential n-3 fatty acids which experience shows can result after administration of dietary fibers, and in particular as an unwanted side reaction in drug treatment of high cholesterol values with statins.

The use of the inventive dietetic foods has a health-promoting effect via the actual cardiovascular health.

Carob products in the context of the invention are the carob fruit itself and also components produced therefrom. Those which are preferably used in the context of the invention are carob fibers which are characterized by a high content of total dietary fibers determined by AOAC method 985.29, of at least 30% by weight, preferably at least 60% by weight, particularly preferably at least 80% by weight (in each case based on the dry mass). Their content of water-insoluble dietary fibers determined by AOAC method 991.42 is at least 25% by weight, preferably at least 50% by weight, particularly preferably at least 70% by weight.

To produce the carob fiber product, in particular removal of the water-soluble carob components from the fruit flesh freed from the carob seeds and heating for the (partial) denaturation of the condensed tannins are necessary. Further process steps comprise washing and separation steps, drying, grinding and appropriate sifting. This produces fiber lengths of <250 μm, preferably <150 μm, in particular <100 μm. Particular preference is given to the methods according to EP-A-0 616 780 and according to the unpublished PCT/EP03/08636. The preparations thus produced exhibit a pronounced hypocholesterolemic action and moderate triglyceride-reducing action and can be used to enrich foods.

For the purposes of the invention, n-3 fatty acids (omega-3 fatty acids, ω-3 fatty acids) are taken to mean long-chain polyunsaturated fatty acids (PUFAs) having a chain length >C12 having at least two double bonds, the first of the at least two or more double bonds being constituted between the carbon atoms C3 and C4, starting from the alkyl end (see table 1). Here, the n-3 fatty acids can be present not only as free fatty acids, esters, triglycerides, phospholipids, glycolipids, sphingolipids, waxes or sterol esters, or can have been enriched in the form of their monohydric alcohol esters by chemical or biocatalytic transesterification of the triglycerides, eg using suitable enzymes (lipases). All of these substances and also products which comprise these substances in concentrations of at least 15 area % of TFA (see below) are summarized hereinafter by the terms n-3 fatty acid or n-3 active compounds; these terms are used synonymously.

TABLE 1
n-3 fatty acids

| | | Trivial name, |
|---|---|---|
| | IUPAC name | abbreviation |
| C18:3 | All-cis-9,12,15-Octadeca- | α-Linolenic acid |
| | trienoic acid | ALA |
| C18:4 | All-cis-6,9,12,15-Octadeca- | Stearidonic acid |
| | tetraenoic acid | |
| C20:3 | All-cis-11,14,17-Eicosatrienoic | |
| | acid | |
| C20:4 | All-cis-8,11,14,17-Eicosa- | ETA |
| | pentaenoic acid | |
| C20:5 | All-cis-5,8,11,14,17-Eicosa- | EPA, timnodonic |
| | pentaenoic acid | acid |
| C22:3 | All-cis-13,16,19-Docosatrienoic | |
| | acid | |
| C22:5 | All-cis-7,10,13,16,19-Docosa- | DPA fish oil w-3 |
| | pentaenoic acid | |
| C22:5 | All-cis-4,7,10,13,16-Docosa- | DPA Protists w-6 |
| | pentaenoic acid | |
| C22:6 | All-cis-4,7,10,13,16,19- | DHA |
| | Docosahexaenoic acid | |

Preference for the purpose of the invention is given to the use of a n-3 active compound having a content of n-3 fatty acids of at least 20 area-% of TFA (area-% relates to the AOCS official method Ce 1b-89; TFA=total fatty acid). Particular preference is given to a content of at least 30 area-% of TFA, in particular of at least 40 area-% of TFA and very particular preference to a content of at least 60 area-% of TFA.

Further preference for the purposes of the invention is given to mixtures of the various n-3 active compounds, preferably at least 2 of the n-3 active compounds DHA, EPA and ALA, and particularly preferably a mixture of the n-3 active compounds DHA and EPA. Very particular preference is given to the use of EPA or DHA as main constituent of the n-3 active compound, in particular the use of DHA as single n-3 active compound.

A suitable source of abovementioned mixture of EPA and DHA is fish oils. A suitable source of ALA is plant oils, in particular linseed oil or hemp oil inter alia.

Particular preference is given to n-3 active compounds which are isolated from microorganisms. Preferred microorganisms are organisms of the Stramenopiles (or Labyrinthulomycota), particularly preferably of the order Thraustochytriales, (Thraustchytriidea), in particular of the genera Schizochytrium, Thraustochytrium and Ulkenia, and also Dinoflagellates (Dinophyta), preferably Crypthecodinium, in particular C. cohnii, which are preferably suitable for producing DHA at a concentration of at least 20 area-% of TFA, preferably at least 30 area-% of TFA, and particularly preferably at least 40 area-% of TFA DHA. In this case, with respect to the production of n-3 fatty acids, the following publications are incorporated in particular by reference: WO-A-91/07398, WO-A-91/11918, WO-A-96/33263 and WO-A-98/03671.

Further suitable sources of EPA and/or DHA are also, eg, microalgae such as Euglena (JP-A-60-196157), Nannochloropsis, Phaeodactylum and others (Tonon et al., Long chain polyunsaturated fatty acid production and partitioning to triacylglycerols in four microalgae. Phytochemistry 2002, 15-24), but also bacteria, preferably eg Shewanella, Vibrio or Moritella (Cho and Mo, Screening and characterization of eicosapentaenoic acid-producing marine bacteria, Biotechnology Letters 1999, 215-218; JP-A-2000/245442; JP-A-2000/245442; JP-A-63-216490, JP-A-2001/309797).

Further possible sources of n-3 fatty acids are transgenic organisms, preferably microorganisms and plants.

In addition, use can be made for the purposes of the invention of n-3 active compounds which are purified or concentrated by various methods known to those skilled in the art (eg chromatography, absorption or adsorption methods, winterization etc) from oils as described above (eg fish oils, vegetable oils or oils from microorganisms).

The inventive dietetic foods comprise a carob product, in particular carob fibers, and at least one n-3 fatty acid. In addition, the agents can comprise customary additives such as solvents, fillers, carriers such as methylcellulose, sweetening carbohydrates and other sweeteners, flavorings, colorants, antioxidants and preservatives.

The combination of carob product and n-3 fatty acids can also be administered in the form of two different administration forms. In this case, suitable food applications for the carob products, in particular the carob fibers, and for the n-3 fatty acids are customary food applications such as bakery products, cereals, snack bars or fruit bars, or drinks powders. Furthermore, direct addition of the carob product and the n-3 fatty acids to self-produced foods and use in form typical of food supplements (inter alia tablets, dragees, hard or soft capsules, sachets, granules, bars etc.) is also possible.

The inventive dietetic foods comprise the food components in amounts which are required to achieve the therapeutic effect in 2- to 4-times daily administration.

The carob product or the carob fiber component is present in the dietetic foods at concentrations which cause a marked cholesterol reduction or affect the HDL/LDL ratio in a beneficial manner. The daily dose of carob fiber can be in the range 1-25 g, customarily 5-15 g.

The n-3 fatty acids are present in the inventive dietetic foods at concentrations which, in the synergy with the above described carob products, cause a marked cholesterol reduction or reduction of the blood fat values, and affect in a positive manner the HDL/LDL ratio. The daily dose of n-3 fatty acids in this case can be in the range from 50 mg to 10 g, customarily from 100 mg to 5 g, and preferably from 200 mg to 2 g.

The intake of the inventive dietetic foods can be taken at one defined daily timepoint, or distributed over the day, the ratios of carob products, in particular carob fibers, and n-3 fatty acid in the intake of relatively small doses corresponding to the abovementioned ratios.

To produce the inventive agents, preferably a process could be followed such that the desired amounts of carob product, in particular carob fibers and n-3 fatty acids are mixed with one another, spray dried, freed from the solvent, agglomerated and/or instantized. Furthermore, all customary methods of food technology or else gallenical production methods such as pressing, kneading or dragee-coating, can be used. The n-3 fatty acids can be added to the mixture in pure form, or encapsulated or microencapsulated, all methods familiar to those skilled in the art such as coacervation, spray drying or fluidized-bed drying being able to be used for the encapsulation or microencapsulation. Inclusion in liposomes or micelles is also possible.

In addition, the n-3 fatty acids can be added to the mixture in a form which permits a continuous release (slow-release) of the fatty acids in the body. Suitable methods for producing these “slow release” formulations are, for example, coating methods, or the use of suitable capsule matrices in (micro)encapsulation.

In addition, use can be made of the inventive carob product itself as carrier or matrix for the n-3 fatty acids.

In the case of the joint administration as claimed in the present invention, it has been found that the combined intake of carob products and n-3 fatty acids leads to a greater reduction of the cholesterol level and of the blood fat values than the sum of the effects when the individual components are administered. It is surprising in this case that the combined administration of carob product, in particular carob fibers, and n-3 fatty acids does not reduce the activity of the individual substances by non-specific interference, but that the observed effects go markedly beyond the effects achievable with individual administration of the substances.

The inventive agents thus permit a greater reduction of the cholesterol level and triglyceride level which is frequently therapeutically desirable, than could previously be achieved by switching over diet or dietetic foods.

Furthermore, the inventive active compound combination can be used in a reinforcing manner in the case of a drug treatment, e.g. with statins, as a result of which the dosage of the drugs can be reduced. In particular, unwanted side effects which frequently occur in the administration of cholesterol-reducing active compounds, can thus be reduced or entirely avoided. The inventive dietetic foods are thus a significant advance in therapy of hypercholesterolemia or hyperlipidemia.

The inventive food components can also be used in a preparation which is suitable for drugs and food supplements and is matched to the optimally acting ratios. For this, e.g. pulverulent or tablet-form preparations for dissolution, but also chewing tablets, come into consideration. These preparations can in addition comprise further constituents (additives) for improving the dissolution, such as soluble carriers, tablet disintegrants, e.g. starch, cellulose, bentonite, pectin or peroxides and carbonates in combination with organic acids and generally colorants, sweeteners such as sucrose, glucose, fructose and other carbohydrates, sugar alcohols, e.g. sorbitol, xylitol, maltitol and isomalt, or sweeteners, e.g. acesulfame-K, cyclamate, saccharin, sucralose or aspartame, and in particular flavorings for improving acceptance.

The inventive agents may be administered in combination, but also separately in the form of a dietetic food or food supplement comprising the carob product, in particular the carob fibers, and a dietetic food or food supplement comprising the n-3 fatty acids. In the case of a combined form, in this case the carob product, in particular the carob fibers, can be used as carriers of the n-3 fatty acids.

The invention is described hereinafter on the basis of examples.

### EXAMPLE 1

Pulverulent preparation (for one portion size)

| | | |
|---|---|---|
| Carob fibers (Caromax ®, Nutrinova, Frankfurt) | 3 | g |
| DHA-rich algal oil (DHA content 43 area-% TFA; | 150 | mg |
| Nutrinova, Frankfurt) | | |
| Xanthan (stabilizer) | 150 | mg |
| Vanillin | 15 | mg |

Suspend the preparation in 150 ml of tepid milk by stirring and drink.

### EXAMPLE 2

Chewing Tablet

| | | |
|---|---|---|
| Carob fiber (Caromax ®, Nutrinova, Frankfurt) | 2 | g |
| DHA-rich algal oil (DHA content 43 area-% TFA; | 120 | mg |
| Nutrinova, Frankfurt) | | |
| Sorbitol | 1.4 | g |
| Magnesium stearate | 15 | mg |
| Acesulfame-K | 12 | mg |
| Aspartame | 12 | mg |

| | |
|---|---|
| Chocolate flavoring | quantum satis |

The chewing tablets are mixed and pressed in a conventional manner.

1). A cholesterol-reducing and triglyceride-reducing agent comprising water-insoluble carob fiber and at least one n-3 fatty acid, the n-3 fatty acid or the n-3 fatty acids being present at a concentration of at least 15 area % of TFA (area % based on the AOCS Official Method Ce 1b-69; TFA=total fatty acid).

2). The agent as claimed in claim 1, wherein the n-3 fatty acid is a polyunsaturated fatty acid having a chain length >C12 having at least two double bonds, or its ester, triglyceride, phospholipid, glycolipid, sphingolipid, wax or sterol ester.

3). The agent as claimed in claim 1, wherein the n-3 fatty acid is selected from one or more of the following substances: all-cis-9,12,15-octadecatrienoic acid (ALA), all-cis-6,9,12,15-octadecatetraenoic acid, all-cis-11,14,17-eicosatrienoic acid, all-cis-8,11,14,17-eicosatetraenoic acid, all-cis-5,8,11,14,17-eicosapentaenoic acid (EPA), all-cis-13,16,19-docosatrienoic acid, all-cis-7,10,13,16,19-docosapentaenoic acid (DPA) and all-cis-4,7,10,13,16,19-docosahexaenoic acid (DHA).

4). The agent as claimed in claim 3, wherein the n-3 fatty acid is all-cis-4,7,10,13,16,19-docosahexaenoic acid (DHA).

5). A cholesterol-reducing combination preparation comprising water-insoluble carob fiber and at least one n-3 fatty acid in separate administration forms, the n-3 fatty acid or the n-3 fatty acids being present at a concentration of at least 15 area % of TFA (area % based on the AOCS official method Ce 1b-69; TFA=total fatty acid).

6). A method for producing an agent as claimed in claim 1, which comprises mixing at least one carob product and at least one cholesterol-reducing active compound with one another.

7). A drug comprising an agent as claimed in claim 1.

8). A drug as claimed in claim 7, wherein said drug is a cholesterol-reducing drug.

9). A drug as claimed in claim 7 wherein said drug is a hypercholesterolemia, hyperlipidemia or arteriosclerosis drug.

10). A drug as claimed in claim 7 wherein said drug postitively shifts the HDL/LDL ratio.

11). An agent as claimed in claim 1, wherein said agent is administered along with an additional supply with n-3 fatty acids.
